(19) United States
(12) Patent Application Publication
(10) Pub. No.: US 2002/0061843 A1
(43) Pub. Date: May 23, 2002

(54) Use of mCRP for delivery of materials into cells

(76) Inventors: Lawrence A. Potempa, Deerfield, IL (US); James A. Radosevich, Rockford, IL (US); Edward E. Diehl, Chicago, IL (US)

Correspondence Address: SHERIDAN ROSS PC, 1560 BROADWAY, SUITE 1200, DENVER, CO, 80202

(21) Appl. No.: 09/971,100

(22) Filed: Oct. 3, 2001

Related U.S. Application Data
(63) Continuation of application No. 09/376,630, filed on Aug. 18, 1999, now abandoned.
(60) Provisional application No. 60/097,128, filed on Aug. 19, 1998.

(51) Int. Cl. A61K 48/00; A61K 38/16; A61K 9/127

(52) U.S. Cl. 514/8; 514/44; 424/450

(57) ABSTRACT

The invention provides a method of delivering a material into a cell. The method comprises associating the material with a modified C-reactive protein (mCRP) or a mutant-mCRP. Then, the material associated with the mCRP or the mutant-mCRP is contacted with the cell so that the material is delivered into the cell.

The invention also provides a kit comprising one or more containers. In one embodiment, one of the containers holds a material to be delivered into a cell in association with a modified C-reactive protein (mCRP) or a mutant-mCRP. Alternatively, one of the containers holds the material to be delivered into the cell, and a second container holds the mCRP or the mutant-mCRP.

This application claims benefit of provisional application No. 60/097,128, filed Aug. 19, 1998.

### FIELD OF THE INVENTION

The invention is concerned with the delivery of materials into cells. In particular, it has been discovered that modified C-reactive protein (mCRP) and mutant-mCRPs can be used to deliver materials into cells.

### BACKGROUND OF THE INVENTION

During injury, invasion of pathogens, or other forms of tissue damage, higher vertebrates implement a cascade of biochemical, immune and inflammatory reactions collectively termed the acute phase response. The inflammation results in an increase in blood flow and the delivery of important factors to the affected site. These factors act to limit microbial growth, reduce tissue damage, and aid in the removal of damaged tissue. The acute phase response is a primitive, nonspecific mechanism which reacts quickly prior to the development of the specific processes of humoral and cellular immunity.

C-reactive protein (CRP) has long been recognized as an important acute phase response protein, and its concentration in serum may increase as much as 1,000-fold during the acute phase response. CRP is a pentamer consisting of five identical subunits, each having a molecular weight of about 23,500. The pentameric form of CRP is sometimes referred to as “native CRP.”

In about 1983, another form of CRP was discovered which is referred to as “modified-CRP” or “mCRP.” The formation of mCRP from native CRP involves the dissociation of native CRP into its subunits which also undergo a change in conformation. As a result, mCRP expresses antigenicity which is distinct from that of native CRP (referred to as “neo-CRP antigenicity”), and antibodies are available which can distinguish mCRP from native CRP (see, e.g., U.S. Pat. No. 5,272,258 and Potempa et al., Mol. Immunol., 24,531-541 (1987)). The conversion of native CRP into mCRP is irreversible (the subunits do not reassemble into native CRP). Kresl et al., Int'l J. Biochem. Cell Biol., 30, 1415-1426 (1998).

It has been reported that mCRP can influence the development of monocyte cytotoxicity, improve the accessory cell function of monocytes, potentiate aggregated IgG-induced phagocytic cell oxidative metabolism, and increase the production of interleukin-1, prostaglandin E and lipoxygenase products by monocytes. Potempa et al., Protides Biol. Fluids, 34, 287-290 (1987); Potempa et al., Inflammation, 12, 391-405 (1988); Potempa et al., Proc. Amer. Acad. Cancer Res., 28, 344a (1987); Chu et al., Proc. Amer. Acad. Cancer Res., 28, 344a (1987); Zeller et al., Fed. Proc., 46, 1033a (1987); Chu et al., Proc. Amer. Acad. Cancer Res., 29, 371a (1988). It is also known that mCRP can be used to treat viral infections, bacterial infections, endotoxic shock and cancer. See U.S. Pat. Nos. 5,283,238, 5,405,832, 5,474,904, and 5,585,349. It is further known that mCRP stimulates thrombocytopoiesis and the maturation of megakaryocytes and that it can be used to treat thrombocytopenia. See U.S. Pat. No. 5,547,931. Finally, it is known that mCRP binds immune complexes and aggregated immunoglobulin and can, therefore, be used to remove immune complexes and aggregated immunoglobulin from fluids and to quantitate immune complexes. See U.S. Pat. No. 5,593,897. It should be noted that mCRP differs from native CRP in its biological activities. See, e.g., the patents listed above.

### SUMMARY OF THE INVENTION

The present invention provides a method of delivering materials into cells using mCRP or mutant-mCRP, both as defined below. To do so, a material is associated with an mCRP or a mutant-mCRP, and the material associated with the mCRP or mutant-mCRP is contacted with the cells so that it is delivered into the cells.

The invention also provides a kit comprising one or more containers. In one embodiment, one of the containers holds a material to be delivered into a cell, and a second container holds an mCRP or a mutant-mCRP. In an alternative embodiment, a single container holds the material to be delivered into the cell in association with the mCRP or the mutant-mCRP.

### BRIEF DESCRIPTION OF THE DRAWING

FIG. 1: A Western blot. A549 pulmonary adenocarcinoma cells were cultured with control buffer (lanes A, B), 150 μg/ml native CRP (lanes C, D) or 150 μg/ml mCRP (lanes E, F). After 48 hours, the cells were harvested with a scraper (lanes A, C, E) or with trypsin (lane B, D, F). Equal amounts of protein were loaded onto an SDS-PAGE, and the Western blot was developed using anti-mCRP monoclonal antibody 9C9.

### DETAILED DESCRIPTION OF THE PRESENTLY PREFERRED EMBODIMENTS OF THE INVENTION

Modified-CRP can be prepared by using native CRP as the starting material. The native CRP used for preparation of mCRP can be obtained from natural sources (e.g., serum, plasma, pleural fluid or ascites fluid). Methods of isolating native CRP from natural sources are known in the art and are described, for example, by Volanakis et al., J Immunol., 113:9-17 (1978); de Beer et al., J. Immunol. Meth., 50:17-31 (1982); Potempa et al., Mol. Immunol., 24:531-541 (1987). CRP is preferably isolated from serum, plasma, pleural fluid, or ascites fluid by calcium-dependent affinity chromatography using phosphorylcholine-substituted BioGel® A 0.5 m (an agarose-based resin obtained from BioRad Laboratories, Richmond, Calif.). See, Potempa et al., Mol. Immunol., 24:531-541 (1987). Using this isolation method, CRP can be obtained which is about 99% pure. Partially purified CRP may be obtained from commercial sources, such as Western States Plasma (Fallbrook, Calif.).

Native CRP can also be produced by recombinant DNA techniques. Genomic and cDNA clones coding for human, mouse, and rabbit CRP have been isolated and sequenced. Tucci et al., J. Immunol., 131, 2416-2419 (1983); Whitehead et al., Science, 221, 69-71 (1983); Lei et al., J. Biol. Chem., 260, 13377-83 (1985); Woo et al., J. Biol. Chem., 260, 13384-88 (1985); Hu et al., Biochem., 25, 7834-39 (1986); Samols and Hu, Protides Biol. Fluids, 34, 263-66 (1986); Syin et al., J. Biol. Chem., 261, 5473-79 (1986); Ciliberto et al., Nucleic Acids Res., 15, 5895 (1987); Hu et al., J Biol. Chem., 263, 1500-1504 (1988); Whitehead et al., Biochem, J., 266, 283-90 (1990). Further, there is substantial homology between the amino acid sequences of CRPs from different species. For instance, there is from about 50% to about 80% sequence homology between CRPs from various mammalian species. Hu et al., Biochem., 25, 7834-39 (1986); Whitehead et al., Biochem, J., 266, 283-90 (1990); and Kilpatrick et al., Immunol. Res., 10, 43-53 (1991). Given the substantial homology between CRPs from different species, probes can readily be prepared from the known clones so that genomic and cDNA clones can be isolated which code for CRP from other species. Methods of preparing such probes and isolating genomic and cDNA clones are well known. See, e.g., Lei et al., J. Biol. Chem., 260, 13377-83 (1985); Woo et al., J. Biol. Chem., 260, 13384-88 (1985); Hu et al., Biochem., 25, 7834-39 (1986); Hu et al., J. Biol. Chem., 263, 1500-1504 (1988); Whitehead et al., Biochem. J, 266,283-90 (1990). To obtain native CRP, eukaryotic host cells, preferably mammalian host cells, should be used for the expression of the CRP clone. See Samols and Hu, Protides Biol. Fluids, 34, 263-66 (1986); Hu et al., J. Biol. Chem., 263, 1500-1504 (1988).

Methods of making mCRP from native CRP are known in the art (See, e.g., Potempa et al., Mol. Immunol., 20, 1165-1175 (1983); Potempa et al., Mol. Immunol., 24, 531-541 (1987)). For instance, mCRP can be prepared by denaturing CRP. CRP can be denatured by treatment with an effective amount of urea (preferably 8 M) in the presence of a conventional chelator (preferably ethylenediamine tetraacetic acid (EDTA) or citric acid). Further, CRP can be treated to produce mCRP by adjusting the pH of the protein to below about 3 or above about 11-12. Finally, mCRP can be produced by heating CRP above 50° C., for a time sufficient to cause denaturation (preferably at 63° C. for 2 minutes), in the absence of calcium or in the presence of a chelator.

Monomeric preCRP, produced by cell-free translation of DNA coding for it, expresses neo-CRP antigenicity. preCRP is a precursor protein consisting of a signal or leader sequence attached to the N-terminus of the CRP subunit. During normal processing, the signal or leader sequence is cleaved from the preCRP molecules to produce mature CRP subunits which assemble into pentameric native CRP. This normal processing and assembly occur in eukaryotic cells. See Tucci et al., J. Immunol., 131, 2416-2419 (1983); Samols and Hu, Protides Biol. Fluids, 34, 263-66 (1986); Hu et al., J. Biol. Chem., 263, 1500-1504 (1988). Therefore, mCRP can be prepared directly by recombinant DNA techniques by selecting conditions so that the CRP subunits are not assembled into pentameric native CRP. This can be accomplished by expressing a desired genomic or cDNA clone in prokaryotic cells (referred to herein as “recombinant-mCRP” or “rₘCRP”). Recombinant-mCRP produced in prokaryotic cells consists of CRP subunits, preCRPs and/or fragments of the subunits and preCRPs. The CRP subunits and preCRPs may have slightly altered N-terminal and C-terminal sequences which reflect or assist their production in prokaryotic cells. For instance, they may have methionine as the N-terminal amino acid.

Therefore, as used herein, the terms “modified-CRP” and “mCRP” mean preCRPs or subunits of CRP, in free or aggregated form, which express neo-CRP antigenicity. The terms comprise all of those forms of mCRP described above, including CRP subunits and preCRPs having slightly altered N-terminal and C-terminal sequences which reflect or assist their production in prokaryotic cells. Neo-CRP antigenicity can be detected using antibodies specific for mCRP (see, e.g., U.S. Pat. No. 5,272,258 and Potempa et al., Mol. Immunol., 24, 531-541 (1987)) in standard immunoassays. Further, given the substantial homology between the amino acid sequences of CRPs from different species, it is expected that mCRP from any species will be effective in the practice of the invention.

To avoid the aggregation of the CRP subunits and preCRPs that generally occurs when DNA coding for preCRP is expressed in prokaryotic cells, mutant CRP subunits and preCRPs have been developed. See U.S. Pat. No. 5,874,238. These mutant CRP subunits and preCRPs contain one or more amino acid changes that produce CRP subunits and preCRPs that are less likely to aggregate when produced in prokaryotic cells. The amino acid(s) added, deleted and/or replaced are also chosen so that the mutant protein retains the neo-CRP antigenicity characteristic of mCRP.

Suitable amino acid changes include the deletion or replacement of at least one, preferably all, of the cysteines in an unmutated CRP subunit or unmutated preCRP. CRP subunits contain two cysteines and preCRP's contain three cysteines, and it is believed that some of these cysteines form intermolecular disulfide bonds, thereby contributing to the formation of non-dissociable cross-linked aggregates. Therefore, one or more, preferably all, of these cysteines are desirably deleted or replaced. When the cysteines are replaced with other amino acids, they are preferably replaced with glycine, alanine, valine, leucine, isoleucine, serine, threonine or methionine, but any amino acid can be used. Most preferred is substitution with alanine. Lysine and derivatized lysine residues may also contribute to non-dissociable cross-linking. Accordingly, suitable amino acid changes may also include the deletion or replacement of at least one of the lysines in an unmutated CRP subunit or unmutated preCRP. As a result of the amino acid changes in them, the mutant proteins are easier to purify with much higher yields than unmutated CRP subunits or unmutated preCRP's.

Not all of the amino acid additions, deletions and replacements need contribute to the reduced likelihood of forming non-dissociable aggregates as long as the combined effect of all the changes is a reduction in intermolecular non-dissociable cross-linking. For instance, the recombinant DNA manipulations used to produce the mutant proteins may result in amino acids being added at the amino or carboxy terminal ends of the CRP subunit. This is acceptable as long as these amino acids do not contribute to the production of nondissociable aggregates. In addition, some of the amino acid changes may be made for other purposes. For instance, it is desirable to make amino acid changes which increase the solubility of the resultant mutant protein in aqueous media, since a more soluble mutant protein is easier to purify and process. Suitable amino acid changes to increase the solubility include deleting one or more hydrophobic amino acids, replacing one or more hydrophobic amino acids with charged amino acids, adding one or more charged amino acids, or combinations of these changes. However, for the reasons stated above, it may be desirable to avoid the addition of lysine residues. Aqueous media include water, saline, buffers, culture media, and body fluids. As another example, amino acid changes can be made for the purposes of providing for the association of the material to be delivered into the cell with the mutated CRP subunit or preCRP (see below).

The mutant proteins can be prepared by expression of DNA coding for them in transformed host cells. DNA coding for a mutant protein can be prepared by in vitro mutagenesis of a known or newly-isolated CRP genomic or cDNA clone or can be chemically synthesized. In vitro mutagenesis techniques are conventional and well known. Particularly preferred is site-directed mutagenesis using polymerase chain reaction (PCR) amplification. See, e.g., U.S. Pat. No. 5,547,931. The following references described other site-directed mutagenesis techniques which can be used to produce DNA coding for a mutant protein: Current Protocols In Molecular Biology, Chapter 8 (Ansubel ed. 1987); Smith & Gilliam, Genetic Engineering Principles And Methods, 3, 1-32 (1981); Zoller & Smith, Nucleic Acids Res., 10, 6487-6500 (1982); Zoller et al., Methods Enzymol, 100,468-500 (1983); Zoller & Smith, DNA, 3, 479-88 (1984); Brake et al., Proc. Natl. Acad. Sci. USA, 81, 4642-46 (1984); Bio/Technology, pages 636-39 (July 1984); Botstein et al., Science, 229, 1193 (1985); Kunkel et al., Methods. Enzymol., 154, 367-82 (1987).

DNA coding for a mutant protein of the invention can also be prepared by chemical synthesis. Methods of chemically synthesizing DNA having a specific sequence are well-known in the art. Such procedures include the phosphoramidite method (see, e.g., Beaucage and Caruthers, Tetrahedron Letters, 22, 1859 (1981); Matteucci and Caruthers, Tetrahedron Letters, 21, 719 (1980); and Matteucci and Caruthers, J. Amer. Chem. Soc., 103, 3185 (1981)) and the phosphotriester approach (see, e.g., Ito et al., Nucleic Acids Res., 10, 1755-69 (1982)).

Therefore, as used herein, the term “mutant-mCRP” means preCRPs or subunits of CRP having a sequence mutated as described above which express neo-CRP antigenicity. As noted above, neo-CRP antigenicity can be detected using specific antibodies in standard immunoassays. Further, given the substantial homology between the amino acid sequences of CRPs from different species, it is expected that mutant-mCRPs derived from the preCRPs or CRP subunits of any species will be effective in the presently claimed invention.

For a detailed description of the physical and chemical properties, biological activities, and methods of making mCRP, including rₘCRP, and mutant-mCRP, and antibodies to neo-CRP antigenicity, see U.S. Pat. Nos. 5,272,258, 5,283,238, 5,405,832, 5,474,904, 5,547,931, 5,585,349, 5,593,897, and 5,874,238, published PCT application WO 94/18999, and U.S. patent application Ser. Nos. 08/480,270, 08/548,974, 08/549,013 and 08/767,795, the complete disclosures of which are incorporated herein by reference.

Fragments of CRP subunits and preCRPs, having a native or mutant sequence, may have the same activities described herein for mCRP and mutant-mCRP, and the use of such fragments is considered to come within the scope of the present invention. It is also believed that proteins substantially homologous to CRP will have the activities described herein for mCRP, and such proteins are also considered to come within the scope of the present invention.

The present invention is based on the discovery that, when mCRP is contacted with cells, it is internalized. Thus, materials can be delivered into cells by associating them with mCRP or mutant-mCRP. Further, the internalized mCRP is distributed in the cells in a pattern which indicates association with intermediate filament cytoskeletal proteins. Intermediate filaments are known to form a link from the extracellular region to the cell nucleus, where they interact with chromatin and help regulate gene activity. Intermediate filaments can extend into the extracellular space through membrane pores such as desmosomes and hemidesmosomes. It is believed that mCRP and mutant-mCRP enter the cells using the intermediate filaments as a conduit. It is expected that the use of mCRP and mutant-mCRP will, therefore, provide a means of delivering materials into the nucleus of cells, as well as into the cytoplasm of the cells.

Materials may be associated with an mCRP or a mutant-mCRP in a variety of ways for delivery into cells. For instance, a material which is to be delivered into cells can be encapsulated in liposomes having the mCRP or mutant-mCRP on their surfaces. In addition, since it is believed that mCRP and mutant-mCRP will be internalized by any type of cell, the liposomes may have other molecules (e.g., antibodies) attached to the surface for targeting the liposomes to the cells to which it is desired that the material be delivered. Methods of making liposomes, encapsulating materials in liposomes, and attaching compounds, including targeting compounds, to the surfaces of liposomes are well known in the art. See, e.g., U.S. Pat. Nos. 5,283,238 and 5,858,399 and references cited therein.

It is known that mCRP is soluble in solutions of low ionic strength and that it aggregates in solutions of high ionic strength. “Solution of low ionic strength” means a solution containing ≦0.05 M NaCl or a solution of another salt having an equivalent relative salt concentration. “Solution of high ionic strength” means a solution containing >0.05 M NaCl or a solution of another salt having an equivalent relative salt concentration, including physiological solutions (about 0.15 M NaCl or a solution having an equivalent salt concentration). Thus, a material can be associated with an mCRP by providing the mCRP in a low ionic strength solution, adding the material to the solution, and increasing the ionic strength ofthe solution so that the mCRP aggregates, trapping the material in the aggregates. The resulting mCRP aggregates will include the material as part of the aggregates. A material can be associated with a mutant-mCRP in this same manner, provided that the amino acid changes in the mutant-mCRP have not substantially changed the solubility of the mutant-mCRP as compared to mCRP. Since mCRP has strong hydrophobic characteristics, a material can be associated with most mutant-mCRPs in this manner.

Neutral materials will associate with mCRP as a result of hydrophobic interactions. Thus, such a material can simply be contacted with an mCRP for a time sufficient (such times can be determined empirically) so that complexes of the material and the mCRP are formed as result of these hydrophobic interactions, and the resulting complexes can be used in the practice of the present invention. The contacting of the material and the mCRP may take place in a solution of low ionic strength or a solution of high ionic strength. A material can be associated with a mutant-mCRP in this same way, provided that the amino acid changes in the mutant-mCRP do not substantially change the hydrophobicity of the mutant-mCRP as compared to mCRP. As noted above, mCRP has strong hydrophobic characteristics, and neutral materials can be associated with most mutant-mCRPs in this manner.

Anionic and cationic materials can be associated with an mCRP which has been altered to be more positively or negatively charged, as a result of ionic interactions. Methods of derivatizing proteins to change their charge are well known. See, e.g., Means and Feeney, Chemical Modification of proteins (Holden-Day Inc., San Francisco, 1971). For instance, positively-charged groups or negatively-charged groups could be attached to the mCRP. Alternatively, the mCRP could be chemically treated to change its charge (e.g., by changing positively-charged residues to negatively-charged residues). In another alternative, mutant-mCRPs mutated to be more positively charged or more negatively charged could be used.

Finally, materials can be associated with an mCRP or a mutant-mCRP by covalent attachment to the mCRP or mutant-mCRP. Methods of covalently attaching materials to proteins are well known. For a description of suitable coupling agents and their use, see Handbook and General Catalog (Pierce Chemical Co. 1989); Catalog And Handbook (Pierce Chemical Co. 1994-1995); Products Catalog (Pierce Chemical Co. 1997); Peeters, et al., J. Immunol. Methods, 120, 133-143 (1989); Pauillac, et al., J. Immunol. Methods, 220, 105-114 (1998); Kitagawa, J. Biochem., 94, 1165-1172 (1983); Bauminger and Wilchek, Methods Enzymol., 70, 151-159 (1980); Erlanger, Methods In Enzymology, 70, 85-104 (1980); Makela and Seppala, Handbook of Experimental Immunology (Blackwell 1986); Parker, Radioimmunoassay of Biologically Active Compounds (Prentice-Hall 1976); Butler J. Immunol. Meth., 7, 1-24 (1974); Weinryb and Shroff, Drug. Metab. Rev., 10, 271-83 (1979); Broughton and Strong, Clin. Chem., 22, 726-32 (1976); Playfair et al., Br. Med. Bull., 30, 24-31 (1974); and U.S. Pat. Nos. 4,990,596 and 4,782,136.

Materials which can be delivered into cells by mCRP and mutant-mCRP include small organic molecules, peptides, proteins, oligonucleotides, nucleic acids, carbohydrates, and pathogens or fragments of pathogens. These materials may function in the cells as drugs, probes and/or may alter or regulate the functioning of the cells in one or more ways. Specific materials which can be delivered into cells include antibiotics, antiviral agents, antifungal agents, growth factors, anti-inflammatory agents (e.g., steroids and nonsteroidal anti-inflammatory agents), oligonucleotide probes, antisense RNA, ribozymes, genes, antibodies (as drugs or probes or which can alter or regulate cell functions), proteins or polypeptides absent or deficient in the cells, physiologically-active peptides (e.g., interleukins and interferons), hormones (peptide and non-peptide), immune modulators, and cytotoxic agents (e.g., toxins and chemotherapeutics) for eliminating diseased or malignant cells. “Drug” is defined herein to be any material which produces a therapeutic effect. In particular, by the method of the invention, drugs which normally have difficulty entering cells may be readily delivered into cells. Also, lower doses of drugs can be used, since the drugs are delivered into cells. The use of lower doses of drugs should result in fewer adverse side effects. Further, the data suggest that mCRP and mutant-mCRP localize first where they are needed (e.g., in diseased areas). Hence, it is believed that diseased cells will receive a drug before healthy cells do, thereby making treatment more efficient and reducing and/or delaying potential adverse side effects.

A preferred embodiment of the present invention is the use of an mCRP or a mutant-mCRP to deliver antiviral drugs into infected cells. Antiviral drugs are often ineffective since they cannot enter the cells where the virus is found. Associating the drug with an mCRP or a mutant-mCRP provides a means of delivering the antiviral drug into infected cells. Viral infections which can be treated according to the invention include infections caused by Retroviridae (e.g., HIV- 1), Herpesviridae (e.g., herpes simplex, varicella zoster, Epstein-Barr virus, and cytomegaly virus), Hepadnarviridae (e.g., hepatitis B), Picomaviridae (e.g., hepatitis A virus and poliomyelitis virus), Orthomyxoviridae (e.g., influenza virus), Flaviviridae (e.g., yellow fever virus and hepatitis C virus), Rubiviridae (e.g., rubella virus), Parmyxoviridae (e.g., measles, parainfluenza, mumps and canine distemper viruses), Rhabdoviridae (e.g., rabies virus), Papovaviridae (e.g., papillomavirus), and Adenoviridae. Antiviral drugs which can be delivered into cells by associating them with mCRP or mutant-mCRP include azidothymidine (AZT), other nucleoside analogs, protease inhibitors, and minor-groove binding dicationic analogs of pentamidine.

Similarly, some bacterial infections are intracellular. These include infections caused by species of Anaplasma, Baronella, Borrelia, Chlamydia, Coryneform Bacteria (e.g., Corynebacterium, Rhodococcus, and Arcanobacterium), Coxiella, Ehrlichia, Legionella, Mycobacterium, Mycoplasma, Myxovirus, Rikettsia, Salmonella, Treponema, and Yersiniosis. Modified-CRP or mutant-mCRP can be used to deliver antibiotics and other antibacterial drugs into cells infected with such bacteria. Antibacterial drugs which can be delivered into cells by associating them with an mCRP or a mutant-mCRP include amoxicillin, azithromycin, ceftriaxone, chloramphenol, ciprofloxacin, clarithromycin, clindamycin, cotrimoxazole, doxycycline, erythromycin, gentamicin, imipenem,josamycin, ofloxacin, penicillin, priftin, pristinamycin, rifampicin, roxithromycin, streptomycin, tetracycline, teicoplanin, and vancomycin.

As described in the Example below, mCRP enters into and slows the growth of cancer cells, but mCRP is not cytotoxic to the cancer cells. Thus, mCRP or mutant-mCRP may be used to deliver toxins, chemotherapeutics, or other anticancer drugs into cancer cells so that the cancer cells are killed. Preferably, in such a case, the mCRP or mutant-mCRP and anticancer drug are targeted so that the anticancer drug is delivered only to cancer cells. Anticancer drugs which can be delivered into cells by associating them with an mCRP or a mutant-mCRP include thiotepa, busulfan, cyclophosphamide, methotrexate, cytarabine, bleomycin, cisplatin, doxorubicin, melphalan, mercaptopurine, vinblastin and 5-fluorouracil. As used herein, the term “cancer” is used in a broad sense and refers to the physiological condition in mammals that is usually characterized by unregulated cell growth. Cancers treatable by the method of the invention include adenocarcinomas, lymphomas, sarcomas, carcinomas and leukemias. “Probe” is defined herein to be any material that interacts with an intracellular component to produce an observable event. Preferably the probe binds to an intracellular component. The probe can be labeled to allow for visualization of the intracellular component to which it binds. Alternatively, the intracellular component can be visualized using a labeled compound which binds to the probe. Methods of making labeled compounds that can be used as probes or which bind to probes are well known in the art. The probes can be used for research or diagnostic purposes. Since mCRP is not cytotoxic, the probes can be used to study living cells or to diagnose disease in living cells, including living cells in vivo. Probes include: oligonucleotides capable of hybridizing with one or more nucleic acids present, or believed to be present, in the cell; drugs that affect kinases or phosphatases to alter cell signalling pathways; drugs that affect nuclear enzymes or DNA functions (telomerases and topoisomerases); alkylating agents and antimetabolites (e.g., methchlorethamine, azathiotrine and methotrexate); and cytoskeleton inhibiting and disrupting agents (e.g., taxol, pacitaxol, phalloidin, colchicine, cytochalasin and vinblastin).

Materials which alter or regulate the functioning of cells can also be delivered into cells by associating them with an mCRP or a mutant-mCRP. Such materials include recombinant DNA molecules which can be used to transform or transfect cells to express a new phenotype (e.g., transformation or transfection of cells by recombinant DNA techniques so that they produce a heterologous protein or for gene therapy), growth factors, antisense RNA (e.g., directed to mRNA coding for a regulatory protein, such as a kinase or G-protein), ribozymes (e.g., directed to mRNA coding for a regulatory protein, such as a DNA-binding protein), and proteins that alter the metabolism of cells (e.g., antibodies to regulatory proteins).

In a preferred embodiment, recombinant DNA molecules are delivered to cells in vitro for transformation or transfection of cells or are delivered in vitro or in vivo for gene therapy. The use of mCRP or mutant-mCRP to deliver a recombinant DNA molecule to cells may be particularly advantageous, since mCRP and mutant-mCRP appear capable of delivering at least some of the recombinant DNA molecules into the nucleus. Methods and materials for transforming or transfecting cells for obtaining expression of proteins and methods and materials for gene therapy are well known in the art. See, e.g., Sambrook et al., Molecular Cloning: A Laboratory Manual (Cold Spring Harbor Laboratories Press 1989); Culver, Gene Therapy: A Primer For Physicians (rev. 2^{nd} ed., 1996), U.S. Pat. Nos. 5,521,291, 5,460,831, 5,559,099, and 5,874,238, PCT application WO 96/14876, Kirshenbaum et al., J. Clin. Invest., 92, 381-387 (1993), Drazner et al., J. Clin. Invest., 99, 288-296(1997), Kitsis et al., Proc. Natl. Acad. Sci. USA, 88,4138-4142(1991), Kass-Eisler et al., Proc. Natl. Acad. Sci. USA, 90, 11498-11502 (1993), Akhter et al., Proc. Natl. Acad. Sci. USA, 94, 12100-12105 (1997).

Materials can be delivered to cells in vitro or in vivo using the method of the invention. Materials can be delivered to any type of cell from, or in, any species of animal.

In vitro delivery can be achieved by simply contacting the cells in a suitable culture medium with the material associated with the mCRP or mutant-mCRP. Effective amounts of the material associated with the mCRP or mutant-mCRP and times of incubation can be determined empirically, and doing so is within the skill in the art. Culture methods and culture media are well known in the art. Materials which are advantageously delivered in vitro include recombinant DNA molecules for transforming and transfecting cells (e.g., to produce a heterologous protein or for gene therapy) and probes for research and diagnostic purposes.

To deliver a material into a cell in vivo, an effective amount of the material associated with an mCRP or a mutant-mCRP is administered to an animal. Preferably, the animal is a mammal, such as a rabbit, goat, dog, cat, horse or human. The mCRP or mutant-mCRP used in a particular species of animal may be from the same species or a different species of animal. For instance, when the animal is a goat, goat mCRP or human mCRP could be used. However, to avoid an immune reaction, an mCRP or a mutant-mCRP from the same species that is to receive the mCRP or mutant-mCRP should be used. For instance, when the animal is a goat, a goat mCRP or a mutant-mCRP derived from goat CRP subunits or preCRPs should be used. The material associated with the mCRP or mutant-mCRP can be administered in any conventional manner, including orally, intradermally, topically, subcutaneously, intramuscularly, nasally, etc.

Effective amounts (effective dosages and number of doses) of the material associated with either an mCRP or a mutant-mCRP which must be administered to an animal can be determined empirically as is known in the art. It is understood by those skilled in the art that the dose that must be administered will vary depending on, for example, the animal that will receive the material associated with the mCRP or mutant-mCRP, the route(s) of administration, the purpose of the administration (e.g., diagnostic or therapeutic, the condition to be treated, etc.), and the age and size of the animal. It is also understood that it likely will be necessary to give more than one dose of the material associated with the mCRP or mutant-mCRP for therapeutic purposes. Administration of the material associated with the mCRP or mutant-mCRP should be continued until an acceptable response is achieved. As noted above, it is expected that lower doses and fewer doses of a material will be needed to achieve the desired therapeutic goal when the material is administered in association with the mCRP or mutant-mCRP, as compared to being administered alone.

Preferably the material associated with the mCRP or mutant-mCRP is administered in a pharmaceutically-acceptable vehicle. Pharmaceutically-acceptable vehicles are well known in the art. For instance, the vehicle may simply be a liquid, such as saline, buffers or an oil. It could also be a biodegradable polymer, such as poly(lactic/glycolic acid) polymer. Gupta et al., Dev. Biol. Stand., 92, 63-78 (1998); Jones et al., Behring Inst. Mitt., 98, 220-228 (1997). The material associated with the mCRP or mutant-mCRP can also be provided in lyophilized form and reconstituted with a liquid, such as water or saline, just prior to use. It will be apparent to those persons skilled in the art that certain vehicles may be more preferable depending upon, for instance, the route of administration and the nature of the material.

The invention further provides a kit. The kit is a packaged combination of one or more containers holding reagents and other items useful for delivering materials into cells. Suitable containers include bottles, vials, test tubes, microtiter plates, syringes, and other containers known in the art. The kit may comprise one container holding an mCRP or a mutant-mCRP and one container holding a material to be delivered into a cell (e.g., a probe for use in a diagnostic assay or a recombinant DNA molecule for transformation or transfection of cells). Alternatively, the kit may comprise one container holding the material associated with the mCRP or mutant-mCRP. The kit may also contain other items which are known in the art and which may be desirable from a commercial and user standpoint, such as diluents, buffers, empty syringes, PCR primers, labeled materials for detecting probes, gauze pads, disinfectant solution, etc.

### EXAMPLE

This example describes the treatment of tumor cell lines with native CRP and mCRP. The results show that the growth rates of the tumor cells were significantly reduced by mCRP, but not by native CRP, and that mCRP was not cytotoxic. After treatment with mCRP, incubation with trypsin and extensive washing, tumor cells retained large quantities of mCRP, demonstrating that mCRP was internalized by the cells. Following immunofluorescence staining, these cells had a distinct cytoskeletal distribution of mCRP antigen. Finally, traditional immunohistochemistry showed that mCRP was a predominant antigen in healthy tissues and that it was lacking in solid tumors.

Native CRP was isolated from pleural or ascites fluid by calcium-dependent affinity chromatography using phosphorylcholine-substituted BioGel® A 0.5 m (an agarose-based resin obtained from BioRad Laboratories) as described by Volanakis et al. (in J. Immunol., 113:9-17 (1978)) and modified by Potempa et al. (as described in Mol. Immunol., 24:531-41 (1987)). Briefly, the pleural or ascites fluid was passed over the phosphorylcholine-substituted column, and the CRP was allowed to bind. Then, the column was exhaustively washed with 75 mM Tris-HCl-buffered saline (pH 7.2) containing 2 mM CaCl₂ until the absorbance at 280 nm was less than 0.02. The CRP was eluted with 75 mM Tris, 7.5 mM citrate-buffered saline (pH 7.2). This high concentration of Tris significantly reduces non-specifically adsorbed proteins which often contaminate affinity-purified CRP preparations. CRP-containing fractions were pooled, diluted three-to-five fold with deionized water, adsorbed to Q-Sepharose Fast Flow® ion exchange resin (Pharmacia), and then eluted with a linear salt gradient from 0-1M NaCl in 10 mM Tris-HCl, pH 7.4. CRP-containing fractions were pooled and re-calcified to 2-5 mM CaCl₂ (by adding a suitable amount of a 1M solution) and applied to unsubstituted Biogel® A 0.5 m column to remove residual serum amyloid P component (“SAP”). Then, the CRP was concentrated to 1 mg/ml using ultrafiltration (Amicon; PM30 membrane) under 10-20 psi nitrogen. A CRP extinction coefficient (mg/ml) of 1.95 was used to determine concentration. Next, the concentrated CRP was exhaustively dialyzed against 10 mM Tris-HCl-buffered saline, pH 7.2, containing 2 mM CaCl₂. This preparation produced a single Mr 23,000 band on SDS-PAGE electrophoresis and was more than 99% free of SAP, IgG and all other proteins tested for antigenically.

To make mCRP, purified native CRP, prepared as described above, at 1 mg/ml was incubated in 8 M ultra-pure urea in the presence of 10 mM EDTA for one hour at 37° C. The urea was removed by dialysis into 10 mM sodium phosphate buffer (pH 7.4) or Tris-HCl buffer (pH 7.2) containing 0.015 M sodium chloride. The mCRP was sterile filtered through a 0.2 micron filter (Gelman, Ann Arbor, Mich.).

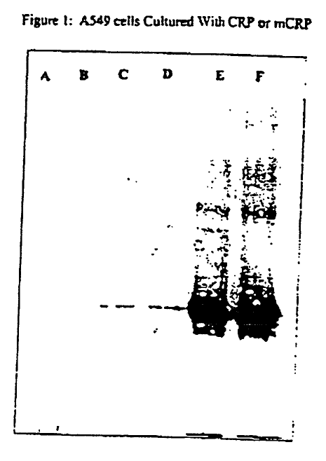

The human pulmonary adenocarcinoma cell line, A549,was obtained from American Type Culture Collection, Rockville, Md. (accession number CCL-185.1). In an experiment designed to determine whether mCRP and native CRP were internalized by the cells, 1.0×10⁶ A549 cells were placed in T25 flasks in 6.0 ml of culture medium (RPMI 1640 with 10% fetal calf serum and supplements) and were incubated overnight under standard conditions (37° C., 5% CO₂) to allow their attachment to the culture flasks. The medium was then aspirated from each flask and replaced with medium containing 150 μg/ml mCRP, 150 μg/ml native CRP, or buffer. Two flasks of each treatment were cultured; one was later used for scraping, and the other was used for trypsinizing, the cells (see below). The flasks were then incubated under standard conditions for 48 hours, at which time the medium was aspirated from each flask. The flasks were washed four times with room-temperature phosphate buffered saline (PBS) by adding the PBS to each flask and allowing the PBS to remain in the flasks for 5 minutes before being aspirated. To prepare “scraped” cells, 6.0 ml of medium were added to each flask, and the cells were scraped with a cell culture scraper to remove them from the flasks. To prepare “trypsinized” cells, 0.5 ml of a dilute trypsin solution was added to each flask, and the flasks were incubated at room temperature for 3-5 minutes, at which time, 6.0 ml of medium were added. The“scraped” cells and “trypsinized” cells were centrifuged and washed twice in room-temperature PBS. Then, the cells were disrupted by sonication (five 30-second bursts with the cells on ice at all times) and centrifuged to remove cell membranes. The protein concentrations of the samples were determined by the Bradford assay, and identical amounts of protein from each of the flasks were loaded onto an SDS-gel. The proteins were transferred to a Western blot and incubated with a monoclonal antibody specific for mCRP (preparation described in U.S. Pat. No. 5,272,258 and Ying et al., J. Immunol., 143:221-228 (1989); routinely antibody 9C9 was used). This monoclonal antibody was detected on the blot using a horseradish-peroxidase-conjugated rabbit anti-IgG antibody. The results are presented in FIG. 1.

The following results were noted:

1) In native CRP-treated cultures, little or no native CRP remained associated with the cells after the above-described treatments.

2) In the mCRP-treated cultures, abundant amounts of mCRP remained associated with cells after trypsin treatment and washing. This finding suggests that the mCRP was taken up by these cells, such that the mCRP was protected from tryptic digestion.

Next, A549 cells were incubated under standard conditions on microscope slides having small reservoirs for culture medium. They were cultured in the presence of 150 μg/ml mCRP or buffer for 24 hours. Antibody 9C9 or a monoclonal that recognizes cytokeratin (an intermediate filament) was then incubated with the cells for 1 hour, after which the slides were washed with PBS to remove excess antibody. Flourescein-conjugated secondary antibodies that recognized monoclonal antibody 9C9 and rhodamine-conjugated secondary antibodies that recognized the cytokeratin monoclonal antibody were then incubated with cells for 1 hour. The slides were washed with PBS to remove excess antibody and examined by fluorescence microscopy. Since each cell had cytokeratin and mCRP labeled, individual cells could be compared with respect to both proteins. This comparison showed that the antibodies to mCRP localized to intracellular fibrous structures that had a distribution pattern similar to that known for intermediate filament cytoskeletal proteins. The distribution of the mCRP antigen was distinct from the distribution of the cytokeratin intermediate filament protein, suggesting that mCRP distributes with one or more intermediate filament protein(s) other than cytokeratin. These data demonstrate that mCRP was not only taken up by the tumor cells, but was also associated with a distinctive cytoskeletal component of the cells.

The highly sensitive metabolic MTT (3-(4,5-dimethylthiazole)-2,5-diphenyl tetrazolium bromide) assay was used to assess cell growth in 96-well plates as previously described in Natarajan et al., BioTechniques, 17:166-171 (1994). The MTT assay simultaneously determines cell count and growth rate. Only living cells take up MTT and reduce it by a mitochondrial dehydrogenase into a colored formazan end product which can be quantified with a spectrophotometer at 540 nM. A549 cells (5000/well) were cultured under standard conditions with 150 μg/ml mCRP, 150 μg/ml native CRP, or buffer (control) for 24 and 48 hours. Native CRP had no effect on cell growth when compared to the buffer control. Modified-CRP, in contrast, reduced the growth rate significantly (a 50% reduction in cell growth rate after 24 hours, and a greater than 70% reduction after 48 hours) and was not directly cytotoxic.

Using a panel of monoclonal antibodies specific for native CRP and mCRP (preparation described in U.S. Pat. No. 5,272,258 and Ying et al., J. Immunol., 143:221-228 (1989)), a wide range of human normal and tumor tissues were assessed using traditional immunohistochemistry, with grading for both the location and intensity of staining.

The tissues examined were:

| | | |
|---|---|---|
| lung adenocarcinoma | thyroid | heart |
| breast - benign | meniscus | adrenal |
| ovarian fibroma | renal cell carcinoma | esophagus - no |
| | | epithelium |
| thyroid goiter | adipose tissue | ovarian tumor |
| small intestine adenoma | colon | liver |
| gastric adenocarcinoma | skin with metastatic | testis |
| ovarian fibroma | lung carcinoma colon | tonsil |
| colon adenocarcinoma | testis | kidney medulla |
| breast adenocarcinoma | uterus | placenta |
| endometrial polyp | placenta | uterus |
| pancreas | uterine leiomyoma | synovial tissue |
| colon adenoma | stomach | gall bladder |
| thyroid | spleen | ovary |
| lung carcinoma | prostate | small intestine |
| kidney | aorta | uterine myometrium |
| ovarian tumor | lung | skin |

It was not known whether or not the immunoreactivity of mCRP and native CRP would be preserved in routinely-prepared, formalin-fixed, paraffin-embedded tissues. Therefore, frozen-unfixed tissues were prepared and used in this study to avoid the chance of having false negative results due to antigen destruction by the fixation and embedding processes. The tissues were handled as follows. Fresh human tissues, usually within 1-2 hours post-excision, were obtained at Northwestern University Medical School, Chicago, Ill. from surgical material not needed for diagnosis or treatment. These tissues were transported to the laboratory on ice, prepared for cryostorage, labelled, and stored in liquid nitrogen (−184° C.) until used. At the time of tissue sectioning, the tissues were warmed to −30° C., cryosectioned, briefly dried at room temperature, cooled to −20° C., and then stored at −70° C.

On several different days (all within a two-week period), sets of coded tissues were immunostained using coded antibodies. Slides with tissue samples were removed from the freezer, air dried for 15 minutes, soaked in acetone for 15 minutes, and washed twice in PBS. Endogenous peroxidases were eliminated by treatment with 3% hydrogen peroxide for 15 minutes. After washing twice in PBS, the slides were incubated with horse serum and 1% bovine serum albumin in PBS, followed by incubation the primary antibodies at 37° C. for 15 minutes. Duplicate slides which were not incubated with monoclonal antibodies served as negative controls. After two washes with PBS, biotin-secondary antibody complexes were added to the slides, which were then incubated at 37° C. for 15 minutes, washed twice in PBS, and incubated with avidin-biotin complexes at 37° C. for 15 minues. After two PBS washes, reactions were visualized by incubation with 3,3′-diaminobenzidine tetrahydrochloride. Slides were then counterstained with hematoxylin.

The various groups of tissues, stained using the monoclonal antibodies, were then reviewed in detail by a trained immunopathologist. The findings can be summarized as follows:

1) Native CRP immunostaining was comparatively rare in normal tissues.

2) Native CRP was very rarely found in tumor tissues.

3) Modified-CRP was frequently found in blood vessels in normal tissues, (significantly higher levels than native CRP).

4) Modified-CRP was not found in the vasculature in and around tumors.

These results support the hypothesis that mCRP is the naturally-occurring, biologically-relevant form of CRP in tissue. These findings further show that mCRP is a predominant antigen in healthy tissues and that it is lacking in solid tumors. The data described above also show that mCRP is taken up by cells and that it associates with the cytoskeleton. These observations suggest a possible link between the extracellular environment and the regulation of tumor growth. The data further show that mCRP enters into and signals tumor cells to reduce their growth rate. Thus, the absence of mCRP in tissues could contribute to the malignant process.

We claim:

1. A method of delivering a material into a cell comprising:
associating the material to be delivered into the cell with a modified C-reactive protein (mCRP) or a mutant-mCRP; and
contacting the material associated with the mCRP or the mutant-mCRP with the cell so that the material associated with the mCRP or the mutant-mCRP is delivered into the cell.

2. The method of claim 1 wherein the contacting takes place in vitro.

3. The method of claim 1 wherein the contacting takes place in vivo.

4. The method of claim 1 wherein the material is a probe.

5. The method of claim 4 wherein the probe is selected from the group consisting of an oligonucleotide, a drug that alters cell signaling pathways, a drug that affects nuclear enzymes or DNA functions, an alkylating agent, an antimetabolite, and a cytoskeleton inhibiting or disrupting agent.

6. The method of claim 1 wherein the material is a drug.

7. The method of claim 6 wherein the drug is an antiviral drug.

8. The method of claim 6 wherein the drug is an antibacterial drug.

9. The method of claim 6 wherein the drug is an anticancer drug.

10. The method of claim 1 wherein the material alters or regulates the functioning of the cell.

11. The method of claim 10 wherein the material is a nucleic acid.

12. The method of claim 11 wherein the material is a recombinant DNA molecule for transformation or transfection of the cell.

13. The method of claim 10 wherein the material is a ribozyme or antisense RNA.

14. The method of claim 1 wherein the material is associated with the mCRP or the mutant-mCRP by encapsulation in a liposome having the mCRP or the mutant-mCRP on its surface.

15. The method of claim 1 wherein the material is associated with the mCRP or the mutant-mCRP by combining the material and the mCRP or the mutant-mCRP is a solution of low ionic strength and then increasing the ionic strength of the solution so that the mCRP or the mutant-mCRP aggregates, the material being trapped in the aggregates.

16. The method of claim 1 wherein the material is associated with the mCRP or the mutant-mCRP as a result of hydrophobic interactions.

17. The method of claim 1 wherein the material is associated with the mCRP or the mutant-mCRP as a result of ionic interactions.

18. The method claim 1 wherein the material is associated with the mCRP or the mutant-mCRP by covalent attachment ofthe material to the mCRP or the mutant-mCRP.

19. A kit comprising one or more containers wherein:
one of the containers holds a material to be delivered into a cell in association with a modified C-reactive protein (mCRP) or a mutant-mCRP; or
one of the containers holds the material to be delivered into the cell and a second container holds the mCRP or the mutant-mCRP.

20. The kit of claim 19 wherein the material is a probe.

21. The kit of claim 19 wherein the material is a recombinant DNA molecule for transformation or transfection of the cell.
